(19) United States
(12) Patent Application Publication
(10) Pub. No.: US 2024/0225925 A1
(43) Pub. Date: Jul. 11, 2024

(54) HYGIENE ARTICLE WITH SUSPENDERS

(71) Applicant: Concepts for Success C4S, Euskirchen (DE)

(72) Inventor: Christoph SCHMITZ, Euskirchen (DE)

(73) Assignee: Concepts for Success C4S, Euskirchen (DE)

(21) Appl. No.: 18/558,657

(22) PCT Filed: May 3, 2022
(86) PCT No.: PCT/EP2022/061745

§ 371 Date: Nov. 2, 2023

(30) Foreign Application Priority Data
May 4, 2021 (EP) 21172125.3
Jan. 26, 2022 (EP) 22153371.4

(51) Int. Cl. A61F 13/74; A61F 13/49; A61F 13/495; A61F 13/512; A61F 13/56

(52) U.S. Cl. CPC A61F 13/74; A61F 13/49007; A61F 13/512; A61F 13/5633; A61F 2013/4908; A61F 2013/4953

(57) ABSTRACT

The present invention relates to the field of hygiene articles, in particular to a means for reducing soiling of the skin of a wearer upon defecation. In particular, it relates to a hygiene article comprising a skin protection sheet that comprises a discontinuity as a faeces passageway, such as an opening or a slit. In use, the sheet and the discontinuity are urged towards the anus by a pull means and spread open by a spreading means, such that faeces can pass through the discontinuity but are separated from the skin of the wearer elsewhere. The pull means of the article is adapted to be connected to a suspender system that allows pulling of the pull means independently from the fixation of the article on the wearer, such that the disposable article and the suspender system represent an improved combined system or kit.

### FIELD OF THE INVENTION

The present invention relates to the field of hygiene appliances, in particular to hygiene articles comprising a means for reducing soiling of the skin of a wearer by body exudates such as faeces, urine, or menses. In particular, it relates to an article with a Skin Protection Sheet that comprises at least one opening in registry with body exudate releasing body openings or genital organs, to which it is urged by a longitudinally acting pull means whilst being spread open cross-directionally by a spreading means, whereby the article is adapted to be combined with suspenders which improve the action of the pull means. Such a hygiene article may suitably be a baby or adult diaper or pant, a training pant, or a menstrual pant.

### BACKGROUND

It is known since long to combine absorbent articles with straps or suspenders for sustained positioning on the body—also well before the era of disposable diapers, see e.g. US852076 (1905) describing an improved suspender for diapers. Actually, such approaches were also suggested to maintain absorbent articles on animals, see e.g. U.S. Ser. No. 10/506,797B2, describing a dog diaper with suspender around dog's waist and chest.

Straps can also be connected to girdles around the waist of a wearer, see e.g. USRe32483 describing an absorbent article comprising a washable moisture impervious flexible holder with an absorbent pad and a waist belt with straps and snap fasteners.

Similarly, GB406158A or U.S. Pat. No. 6,520,947 (Willms) describe diapers with releasably attachable suspender around the waist of a wearer. Also, WO2008026089 (K-C) shows suspender around the waist with length adjustable and refastenable straps.

Other approaches provide straps of the breeches or braces type which run over the shoulders or the neck, e.g. U.S. Pat. No. 1,010,703, for holding diaper and stockings simultaneously.

U.S. Pat. No. 3,729,004 describes a napkin including a layer of absorbent material between an outer impervious sheet and an impervious material strip, which has two apertures spaced on the longitudinal axis to allow urine and excrement to pass separately through to the absorbent layer.

However, modern diaper designs including better fit features around the waist and other features to prevent leakage rendered such straps unnecessary, see exemplarily for a large number of designs EP1247506B1, all aiming at approximating the flat structure of absorbent articles to the three-dimensional shape of a body by stretchable or elastic materials around the waist and legs.

Yet a further approach aims at minimizing the need for elastic features by adopting principles of tailoring, especially darts in the waist region, see e.g. EP25039731B1 (C4S), providing significant fit improvements which are further sustained over the wearing period.

In particular, the precise positioning of the article on the body allows to address a further problem which is hitherto unsatisfactorily, if at all, addressed in the hygiene industry, namely the handling of facces, see e.g. EP2424479B1 (C4S), describing inter alia a faeces separation sheet with an opening that remains in registry with the anus.

Whilst the solution described therein provides a significant improvement over conventional articles, it is still not fully satisfactory. As described in PCT/EP2019/073716 (C4S), unpublished, hereinafter referred to as PCT 716, it has been found that one draw back of the solution of EP2424479B1 is that the facces separation sheet spans from one buttock to the other buttock over the anal cleft. Thus, the solution as described in PCT 716 addresses this by providing “pull means strips” that draw the separation sheet into the anal cleft whilst spreading means maintain the openings cross-directionally open to allow facces to pass through. As described the pull means strip can be tightened by two approaches, as may also be combined. In a first one, the pull means strip is permanently attached in the waist region to portions of the article that are unfolded laterally outwardly upon donning, such that the lateral movement automatically foreshortens the length directional extension and thusly forces the pull means and the separation sheet into the body cleft. In a second one, the pull means strip is manually pulled upon donning and affixed, e.g. by attachment means, laterally outwardly to the article. However, under certain circumstances, e.g. when the article is significantly loaded, these solutions may not maintain the article's position on the wearer as desired.

The present invention provides an alternative for maintaining the pull means strip in the appropriate positioning during use, even if the product is significantly loaded. To this end, the pull means strips are exclusively or additionally connected to one or more straps, that are connected to suspender means, as may be positioned around the hips, and/or around the neck or the shoulders of the wearer.

As the above referenced documents also allow for a further opening in the separation sheet in registry with genitals or other bodily exudates releasing organs also the present invention provides an approach for such designs.

### SUMMARY

The present invention is a hygiene article for being worn on the lower torso of a wearer adapted to receive and retain bodily exudates, which exhibits a length/longitudinal/x-direction and a longitudinal centerline, a width/cross-directional/y-direction, perpendicular thereto and corresponding to the left-right direction of the wearer, and a thickness or z-direction perpendicular to both. The article comprises, relative to a wearer during its intended use, a rear waist region and a front waist region, and a crotch point region positioned longitudinally between the waist regions, comprising a crotch point positioned between the anal opening and the genital organs of a wearer during its intended use. The article is adapted to be converted from a manufacturing configuration into an in-use configuration, wherein the article adopts a general U-shape by the longitudinally extending center line extending from the front or rear waist region of a wearer through the crotch point region into the opposite waist region of the wearer, and wherein the front and rear waist regions are adapted to encircle the waist of a wearer. Further, the article comprises a skin protection sheet (SPS) adapted to separate bodily exudates from the skin of a wearer, and comprising at least one opening adapted to be positioned in registry with a bodily exudate releasing body opening or a genital organ, extending from the front or the rear waist regions at least into the crotch point region of the article, optionally into the opposite waist region. Further, the article comprises a backsheet adapted to retain the bodily exudates, optionally side panels, optionally an absorbent core adapted to absorb liquids of the bodily exudates, and optionally an exudate separation sheet (ESS) adapted to separate exudates from each other or from predetermined portions of the article. At least one of the SPS and the ESS, if present, comprise(s) longitudinal side margins that are overfolded along a longitudinal fold line towards but not over the longitudinal center line of the article. Even further, the article comprises a spreading means (SM) adapted to maintain at least one of the opening(s) cross-directionally open during the article's intended use, a pull means (PM) comprising at least one longitudinally extending pull means strip(s), optionally comprising PM sub-strips, adapted to urge the SPS into the anal cleft of a wearer during the article's intended use, and one or more suspender connecting means adapted to be connected to a suspender system which is connectable to but separate from the article.

The suspender connecting means of the PM strip(s) of the hygiene article may be positioned at the front and/or rear ends of the pull means strip strip(s), or may be positioned in the front and/or rear waist region.

For a hygiene article according to the present invention, the at least one pull means strip may extend from at least one of the cross-directionally extending margins in the front or rear waist region of the SPS or ESS towards the crotch point, optionally further towards or into the opposite waist region, and if covering the opening(s) of the SPS, further comprises (a) pull means opening(s) in registry with the openings(s) of the SPS.

Optionally, the spreading means (SM) being unitary with the pull means, wherein the spreading means and the PM are
partially cut from the pull means by a separation line extending from and intersecting a longitudinal side margin of the pull means towards but terminating at a termination point before intersecting the pull-means opening, whereby the intersection of the longitudinally extending side margin is distanced further away from the crotch point than the termination point of the separation line,
connected at its laterally outward end to the overfolded portions of the SPS or ESS: or
a reinforcement means adapted to maintain a pre-cut opening cross-directionally open, optionally comprising an attachment means, optionally of the skin adhesive type.

Optionally, the PM may comprise a first pull means strip permanently connected to the article in at least one of the waist regions, and a second pull means strip positioned z-directionally adjacent to the first pull means strip comprising suspender connection means.

In another aspect, the present invention is a kit of such a hygiene article and a suspender system.

The suspender system, optionally being re-usable, may be selected from the group consisting of
a waist girdle;
a braces system running over the shoulders of a wearer;
a neck holder system, whereby each of these systems are connected to the article by suspender straps that are adapted to be refastenably connected to the suspender connecting means of the pull means strips of the article.

Optionally, a kit of a disposable article and a suspender system may further comprise a sensor system comprising a sensor element, an energy supply, a connecting element, and a signal transmitter, whereby at least the energy supply and the signal transmitter are connected to or integral with the suspender system. The sensor element may be adapted to detect physical, chemical, or biological characteristics of the disposable article or of exudates deposited therein.

### BRIEF DESCRIPTION OF THE FIGURES

FIG. 1 depicts generally the lower torso of a wearer.

FIG. 2A to C depict how a prior art article with a pull means fits on the lower torso of a wearer.

FIG. 3A to C depict prior art articles for explaining the pull means principle as also applied in the present invention.

FIGS. 4A and B depict alternative executions of the element according to FIG. 3.

FIG. 5A to G depicts an article with pull means strips being connected to the article itself, as known in the art.

FIG. 6A md B describe a difference between a prior art article (FIG. 6A) and one according to the present invention (FIG. 6B).

FIGS. 7A and B depict an article and a suspender system of the waist girdle type, according to the present invention.

FIG. 8A to D describe an article and a suspender system of the braces type, according to the present invention.

FIG. 9A to C describe an article and a suspender system of the neck holder type, according to the present invention.

FIG. 10A to D depict particular executions of the connection between the pull strips of the article and the suspender means, according to the present invention.

FIG. 11A to C depict a particular execution of a pull means system according to the present invention.

FIG. 12 depicts a combination of several articles with a smaller number of suspender in a package.

FIGS. 13A and B depict particular executions of the kit of a disposable article and a suspender according to the present invention.

Figures are schematically only and not scaled Same numerals in various figures refer to the same elements or features, apostrophes indicate multiple executions of the same element or features, e.g. left-right elements.

### DETAILED DESCRIPTION

The present invention relates to a hygiene article comprising a skin protection sheet, hereinafter abbreviated as “SPS”. Such an article of manufacture is typically applied to the lower torso of a human, aiming at reducing soiling of the skin by body exudates, such as during menstruation or when a wearer is not able to control urination or bowel movement.

Such hygiene articles further comprise a backsheet that retains the body exudates and prevents soiling of the environment, such as clothing or bedding. Often such articles also exhibit liquid absorbency.

Optionally, a hygiene article according to the present invention may comprise an exudate separation sheet (ESS), which may separate exudates from each other, such as when contact of urine and faeces should be prevented, or from predetermined portions of the article, such as when a portion particularly adapted to receive urine should not be contaminated by faeces that may impede urine absorption. Thus suitable hygiene articles may be diapers for babies or adults, be these of the “open type” with closure means such as tapes, or be these of the “pants type” closed at the sides so as to create a belt like system around the waist, training pants or fixation pants made of film or other materials.

Hygiene articles may be disposable, i.e. discarded and/or further treated in an environmentally friendly and sound manner and may also be made from or comprise re-usable materials. When an article according to the present invention is combined with a suspender system, as described herein below, either of both may be disposable or re-usable, which may include to be washable, or adapted for a limited number of re-uses.

A hygiene article according to the present invention can be in a “manufacturing configuration” which refers to a state of being produced in a manufacturing system, preferably a high speed manufacturing system, allowing to produce more than hundred pieces per minute, more than 300 pieces per minute, even more than 600 pieces per minute, or even more than 1000 pieces per minute. At the end of the manufacturing line, the hygiene article may be brought into a “packaging configuration”, such as by being cut and folded and/or being combined with a suspender system, in which it is delivered to a user, as may be the wearer or a caretaker, who will then bring the article into a “pre-use configuration” such as by unfolding it. Upon donning on a wearer, the article takes an “in-use configuration”.

Typically, an absorbent article exhibits a width or cross-direction or y-direction, corresponding to a left-right orientation of a user during its intended use. Further, the length, longitudinal or x-direction of the article extends perpendicularly thereto, and in an in-use configuration from a first waist region, e.g. the rear waist region, through the crotch region to the opposite waist region, whereby the respective regions of the article correspond to the body regions of a wearer. Thus, in this in-use configuration the longitudinal center line of an article will take a U-shape configuration, whilst in a manufacturing configuration the longitudinal center line is often a straight line, at least until the end of the manufacturing line, where it might be folded. Further, the article exhibits a thickness or z-direction, perpendicular to the x-, and y-directions. The overall length of the article in an in-use configuration corresponds to the outermost line of the article, which is the backsheet as described below, following this U-shape, thus stretching from the rear waist margin of the article through the crotch region to the front waist margin. In a manufacturing configuration, where individual articles are not yet separated but represent an essentially endless sequence of connected article precursors, the overall length of an article is considered to correspond to the distance of marked-up or imaginary lines where the articles are separated from adjacent ones towards the end of the manufacturing process.

The present invention can be based on the principles of a hygiene article for being worn on the lower torso of a wearer, as described in the above referenced PCT '716 application.

Thus, it may comprise
a skin protection sheet (SPS) adapted to separate bodily exudates from the skin of a wearer,
a backsheet adapted to retain the bodily exudates,
optionally side panels,
optionally an absorbent core adapted to absorb liquids of the bodily exudates, and
optionally an exudate separation sheet (ESS) adapted to separate exudates from each other or from predetermined portions of the article.

At least the SPS comprises at least one opening adapted to be positioned in registry with a bodily exudate releasing body opening or a genital organ. The article further comprises a pull means adapted to urge the SPS into the anal cleft of a wearer during the article's intended use, and spreading means adapted to maintain at least one of the openings cross-directionally open during the article's intended use.

Such a hygiene article may be worn on the lower torso of a wearer and be adapted to receive and retain bodily exudates. During its intended use, the article comprises, relative to a wearer, a rear waist region and a front waist region, each comprising cross-directionally opposite first and second side panel sections and a center section there between, and a crotch point region positioned longitudinally between the waist regions, and comprising a crotch point positioned between the anal opening and the genital organs of a wearer.

The article is adapted to be converted from a manufacturing configuration into an in-use configuration, wherein the hygiene article adopts a general U-shape along the longitudinally extending center line extending from the front or rear waist region of a wearer through the crotch point region into the opposite waist region of the wearer, and wherein the front and rear waist regions are adapted to encircle the waist of a wearer.

The article comprises a skin protection sheet (SPS) comprising an outer SPS surface intended to be in direct contact with the skin of a wearer at least in portions of at least one of the front and rear waist portions, and an opposite SPS surface. The SPS comprises at least one SPS opening adapted to be in an in-use configuration in registry with a bodily exudate releasing body opening or a genital organ and is extending from the front or the rear waist regions at least into the crotch point region of the article, optionally into the opposite waist region.

The article may optionally comprise an Exudate Separation Sheet (ESS) positioned z-directionally towards the opposite SPS surface, being connected to the SPS directly or indirectly at least in the crotch point region and extending from the crotch point region towards at least one of the front and rear waist regions.

The backsheet (BS) is adapted to retain bodily exudates in the article and positioned opposite of the outer surface of the SPS. Side panels (SP) may extend laterally outwardly of the SPS at least in an in-use configuration. Often, though not necessarily, an absorbent core may be positioned z-directionally between the SPS and the backsheet. The design of the absorbent core is not critical for the present invention and it may comprise liquid absorbent material and at least partially liquid permeable envelope web(s). The combinations of certain elements may form precursor or precursor webs, such as the absorbent core or a center piece comprising at least a BS and side panels, that may be unitary with the backsheet or separate and connected thereto.

In the manufacturing configuration, at least one of the SPS and the ESS comprise(s) longitudinal side margins that are overfolded along a longitudinal fold line towards but not over the longitudinal center line of the article.

Further, the article comprises pull means, positioned essentially along the longitudinally extending center line and against the upper or opposite surface of the SPS. Thereby, the PM extends from at least one of the cross-directionally extending margins in the front or rear waist region of the SPS or ESS towards the crotch point, optionally further towards or into the opposite waist region, and if then covering the opening(s) of the SPS, further comprises (a) pull means opening(s) in registry with the opening(s) of the SPS.

Further, the article comprises a spreading means which may be unitary with a pull means strip. Then, a pull means strip may be directly connected to the overfolded portions of the SPS or ESS aside the SPS opening(s). Optionally, the pull means strip comprises a separation line extending from and intersecting a longitudinal side margin of the pull means towards but terminating at a termination point before intersecting the pull-means opening, whereby the intersection of the longitudinally extending side margin is distanced further away from the crotch point than the termination point of the separation line and connected at its laterally outward end to the overfolded portions of the SPS or ESS.

In an in-use configuration, the SPS is lifted z-directionally and adapted to fit into the anal cleft of a wearer by the PM, and the at least one opening of the SPS is cross-directionally maintained open by the SM.

Preferably, the ESS may be selected from the group consisting of hydrophilized nonwoven material, hydrophobic nonwovens, films, and apertured films, or combinations thereof.

For such a hygiene article in a manufacturing configuration, the PM exhibits a cross-directional extension that is larger than the cross-directional distance of the overfolded longitudinal extending side margins of the center piece, whereby the SPS or the ESS is connecting to the upper surface of the PM or SPS in the proximity of the at least one discontinuity. Optionally, and for certain executions preferably, at least the SPS, PM, SM are essentially non-elastic.

The precursor of a hygiene article may—in a manufacturing configuration—exhibit an overall article length corresponding to the one of the article in a manufacturing configuration and comprise sections that are separated by characteristic points along the longitudinal center line, wherein the distances between the characteristic points exhibit the following ranges, whereby the specific point A to F are discussed in more detail herein below:
from the rear waist margin (point A) to the forward end of the waist separation line (point B): 10% to 25%;
from the forward end of the separation line (point B) to the midpoint point of the rear discontinuity (point C): 20% to 40%;
from the midpoint point of the rear discontinuity (point C) to the midpoint point of the front discontinuity (point D): 15% to 25%;
from the midpoint point of the front discontinuity (point D) to the rearward end of the front waist separation line (point E) to the front: 5% to 25%;
from the rearward end of the front waist separation line (point E) to the front waist margin (point F): 10% to 25%,
   whereby the respective percentage figures should add up to 100% corresponding to the overall article length and whereby, when certain features a not present, the distances are counted to the next characteristic point.

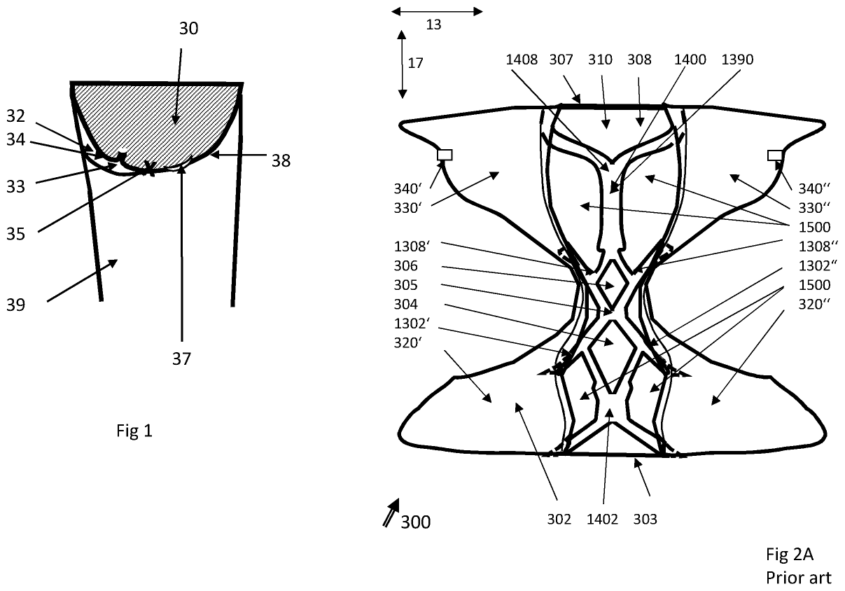

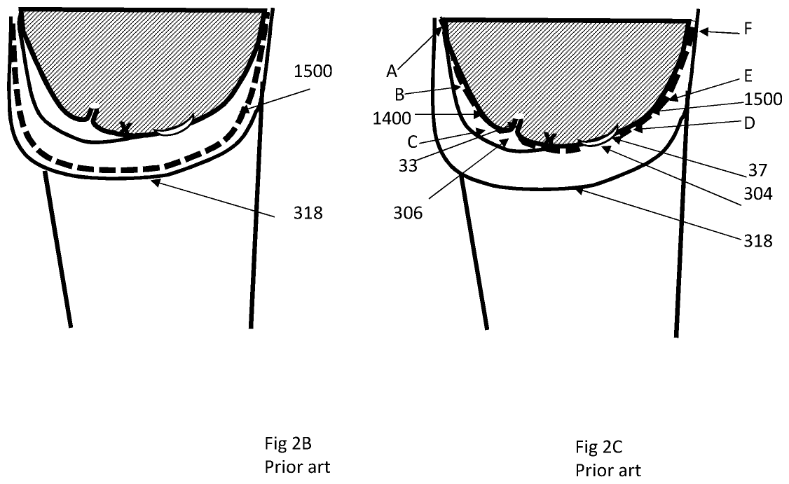

Certain aspects of the present invention, especially the interaction of pull means and spreading means during use, are analogous to the ones of PCT 716, further explained by referring to the FIGS. 1 and 2B, C. FIG. 1 depicts schematically a portion of the sagittal plane of a body, with lower torso 30, upper thigh of a leg 39 and buttocks 32, the latter being separated by the anal cleft 34. Further indicated are the anus 33 and the genital organs, here shown as labium 37, which is also the location of the male genital organs, scrotum and penis. Left and right groin clefts 38 extend from the crotch region forwardly. The perineum with a crotch point 35 is the region between anus and genital organs.

The length of the perineal region has a person to person variability, and is also somewhat dependent on the gender and age. Typically, a range of 2.5 cm and 7 cm covers most of the adults, with a medium length of between 4 cm and 5 cm, both ranges being applicable to female and male persons.

For Babies, the perineal length is typically shorter, though not below about 1 cm. Thus, typically, the crotch point on a wearer is positioned between about 0.5 cm and about 4 cm forwardly of the forward end of the anus.

The crotch point of an article 305 corresponding to the crotch point of a wearer 35 can be determined by placing an article on a wearer of the physical size for which the article is designed and who is in a fully upright standing position with his or her feet a shoulder width apart and then placing an extensible filament around the upper thighs in a figure eight configuration. The point in the article corresponding to the point of intersection of the filament is considered to be the crotch point of the article. Further, the crotch point region of an article is longitudinally extending forward and rearward of the crotch point corresponding to the perineal region of a wearer. In absence of concrete figures for a particular user, the crotch point region is considered to extend at least 1 cm forward and rearward of the crotch point.

Further, FIG. 2A depicts an execution of an article 300 as known from PCT 716 in a pre-use configuration, with longitudinal direction 13 and width direction 17. The article 300 comprises a front region 302 with front cross-directionally extending margin 303 and a rear region 308 with rear cross-directionally extending margin 307 and a crotch region 305 longitudinally there between.

Further, front side panels 320′ and 320″ and rear side panels 330′ and 330″ extend laterally outwardly from a center piece 310. The side panels may be separate materials as connected to the backsheet material, or they may be lateral extensions of the backsheet material.

Generally, when reference is made to features, that are essentially symmetric to a longitudinal center line, as may be a “left-right” symmetry for an article, here shown for the side panels, the respective “left” or “right” features are denoted with single and double quotation marks, respectively, whilst in a general discussion of the feature, no quotation mark is used. The side panels may comprise closure means 340′ and 340″ that allow fitting of the article around the waist of a wearer by connecting front and rear side panels, respectively. As most of the materials suitable for being used in this context are essentially flat or web materials, they generally exhibit two surfaces separated by the thickness.

Within the present context, a first surface of the materials is generally oriented towards the wearer and the second or opposite surface away from the wearer or outwardly, at least in the crotch region, and even if certain portions of the materials are overfolded or are positioned towards the legs of a wearer.

In this exemplary execution of PCT′716, the article 300 further comprises a rear or anal opening 306 and a front or genital opening 304, both being cross-directionally spread open by spreading means (SM) 1300. As will be discussed in more detail herein below, each of the spreading means comprises a pair of spreading elements, here shown for the front, 1302′ and 1302″, and the rear 1308′, 1308″.

Pull means (PM) generally depicted as 1400, here shown as a pull means strip 1410 with a rear pull means 1408 and a front pull means 1402, are indicated as exhibiting a Y-shape, with the stem of the “Y” directed towards the openings 306 and 304, respectively, and the legs of the “Y” directing away from the opening and laterally outward. As shown, the pull means and the spreading means may be unitary. e.g. by being made of a single piece of material. A Skin Protection Sheet (SPS) 1500 is shown in the region of the center piece 310 except for the openings.

FIG. 2B depicts the portion of the sagittal plane of a body as in FIG. 1 with such an article in a pre-use configuration as shown in FIG. 2A with the side panels not being fully outwardly folded and openings, PM, and SM omitted. The SPS 1500 and the backsheet 318 exhibit essentially the same longitudinal extension. Upon donning, and as depicted in FIG. 2C, the outward folding and spreading of the sidepanels for closure of the article around the waist pulls the front and rear ends of the legs of the Y-shaped pull means 1402 and 1408 laterally outward, thusly foreshortening the available longitudinal extension, and thusly urging the pull means 1400 as well as the SPS 1500 connected thereto into the anal cleft such that the openings are also urged towards the anus and genital organs whilst the backsheet remains spaced apart. At the same time, the spreading means induce a lateral pull to the openings thereby widening their CD-extension at predetermined size, such that the rear opening fits tightly to the anus and the front opening is adapted to allow the genital organs to pass through. Thus exudates pass through the openings into the space created between the SPS and the backsheet, separated from the skin by the SPS.

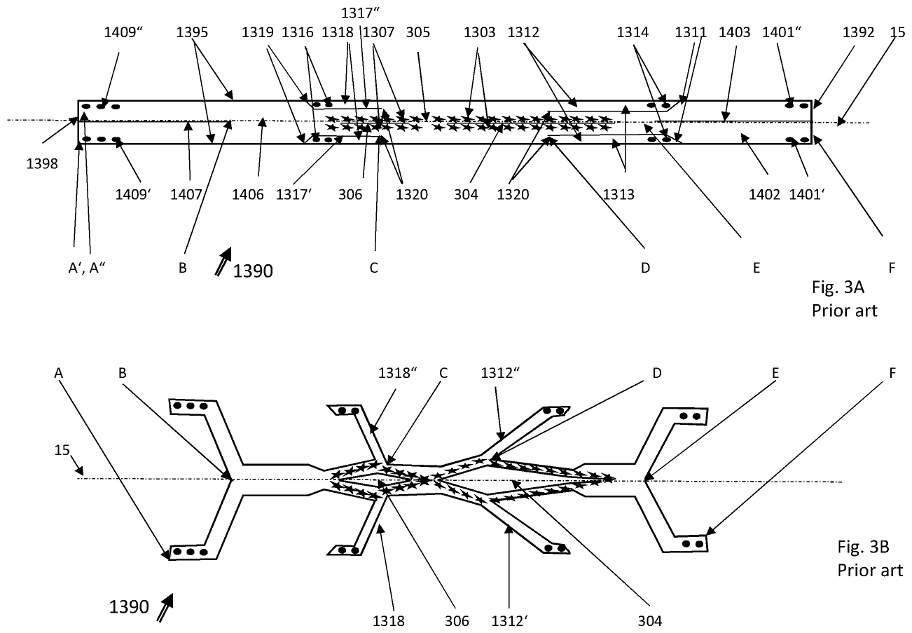

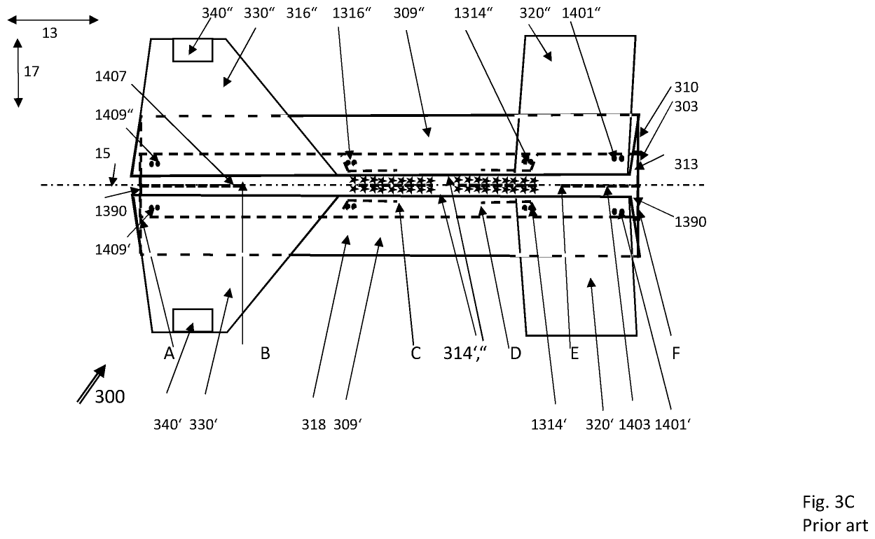

FIGS. 3A and B now refer to features of the pull means 1400 and spreading means 1300, as indicated in the above in the context of FIG. 2 for the particular execution of these being unitary. i.e. made of a single piece of material, as may be referred to as Combined Pull and Spreading Means CPSM 1390.

In FIG. 3A it is shown in a manufacturing configuration, whilst FIG. 3B depicts schematically an in-use configuration (not showing the three-dimensional U-shape), with a view on the wearer oriented first surface. A simple strip of skin friendly material, such as conventional nonwoven material of sufficient strength is provided with a plurality of discontinuities or separation lines, such as may be full cut lines or tear open lines (“perf” n pop).

Front (1402) and rear (1408) pull means comprise discontinuities 1403, 1407, respectively, that extend from the front (1392), respective rear (1398) margins of the CPSM, also indicated by point F and A, respectively, and coinciding with the front and rear margins of the article (shown in FIG. 2A. 303 and 307, respectively). The discontinuities stretch towards the crotch point 305 and stop at points E and B, respectively, corresponding to the node points of the Y-structures described in the above.

The stems of the Y-structures. 1404 and 1406, respectively, further extend from the node points E and B in the direction towards the crotch point up to the discontinuities for the openings 304 and 306.

These discontinuities extend further in the direction towards the crotch point 305, but will not reach there, such that an unseparated region remains in the crotch point region.

For the particular execution as shown in FIGS. 2 and 3, spreading means 1300 are executed as pairs of partially separated strips 1312′ and 1312″ for the front opening 304, and 1318′ and 1318″ for the rear opening 306. These strips are partially separated from the CPSM material by separation lines 1313, 1317, that extend from and intersect a longitudinal side margin 1395′, 1395″ of the CPSM towards but terminating before intersecting the discontinuity for the openings 304, 306, respectively, whereby the intersection with the longitudinally extending side margins and thus the laterally outward end portions 1311′, 1311″, 1319′, 1319″ of the spreading means strips 1312, 1218 are distanced further away from the crotch point 305 than the termination points C and D that are positioned just laterally outwardly of the openings and about midways of the length of the respective discontinuities (as indicated for point C) or more towards the crotch point (as indicated for point D).

In addition to these discontinuities, the CPSM comprises connection points for connecting to other elements of the hygiene article.

In this prior art execution of PCT 716, the front pull means connecting regions 1401′, 1401″ near the front margin 1412 and corresponding to point F, and the rear pull means connecting regions 1409′, 1409″ near the rear margin 1418 and corresponding to point A are positioned laterally outward of the discontinuities 1403 and 1407, respectively, and connect to overfolded portions of the center piece and to the side panels, as will be discussed in more detail herein below.

Similarly, the spreading function is enabled by connecting the laterally outward end portions 1311′, 1311″, 1319′, 1319″ in respective connecting regions 1314′, 1314″, 1316′, 1316″ to overfolded portions of the center piece and to the side panels, as will be discussed in more detail herein below.

Further, the peripheries of the discontinuities forming opening 304 and 306 are connected to the underlying portion of the SPS, which comprises corresponding discontinuities, as will also be discussed herein below.

As indicated in FIG. 3C, a hygiene article 300 comprising a CPSM 1390 is shown in a manufacturing configuration, corresponding to an article as shown in FIG. 2A in a pre-use configuration. The exemplary article comprises a front side panels 320′, 320″, rear side panels 330′, 330″ with closure means 340′, 340″, and a center piece 310. The center piece 310 comprises at least a SPS sheet 1512 as described in the above, and a backsheet 318, typically, though not necessarily an absorbent core (not shown), and exhibits an overfolded “C-shape”, with longitudinally extending side margins 314″, 314″ that are overfolded along longitudinal fold lines 316′, 316″ towards but not over the longitudinal center line 15 of the article, such that in the overfolded portions 309 the backsheet 318 faces the viewer. The center piece 310 exhibits a first surface 313, as may also be referred to as topsheet side, that apart from overfolded regions generally faces towards a wearer in the in-use configuration, and in the manufacturing configuration of FIG. 3C it is merely visible at the front margin 303. In the execution as exemplarily shown in FIGS. 2 and 3, this surface is of the SPS as described above. The side panels are connected to these overfolded portions in proximity of the longitudinal side margins 314, and may—in another variant of a manufacturing configuration—also be downwardly folded around the longitudinal fold line of the center piece, such that this manufacturing configuration is essentially rectangular.

Generally, an article may further comprise various other functional or aesthetic elements, such as side panel extensions, barrier leg cuffs, leg hoops, leg elastics, waist elastics, landing zones and related fasteners, lotions, printing, and other elements as used in products currently available for purchase, all well known as such in the art.

The CPSM strip 1390 is positioned on the first surface of the center piece or to the second surface of the SPS prior to the execution of the overfolding, with the separation lines 1313, 1317, 1403, 1407 cut or otherwise applied thereto as described in the above. Connecting means such as glue or melt-fusion bonding dots or lines can be applied to connecting regions 1303, 1307 in the periphery of the discontinuities for the openings 304 and 306 onto the first surface 313 of the center piece or the corresponding regions of the CPSM, i.e. opposite of the user oriented surface 1397 of the CPSM, such that when the CPSM is placed onto the center piece the connection is established, optionally enhanced by compression, e.g., by pressure roller. Further, connecting means such as glue dots or lines can be applied to connecting regions 1314, 1316, 1401, 1409, or to the corresponding regions on the first surface of the center piece, such that upon overfolding the connection is established, optionally enhanced by compression, e.g., by pressure roller. Thus, in this manufacturing configuration, the CPSM is connected to the center piece
in central portions in the periphery of openings 304 and 306, that are in registry with the SPS and the CPSM, and
along the longitudinal side margins of the overfolded portions.

The principles of such an article as known from PCT′716 can now be explained by considering the conversion from the manufacturing configuration, see FIGS. 3A and C to a pre-use configuration as in FIGS. 2A and B to an in use-configuration as shown in a simplified view for the CPSM in FIGS. 2C and 3B:

When the side panels are pulled laterally outwardly, also the points A′, A″, where the side panels are connected to the CPSM are pulled laterally outwardly, and the legs of the “Y” of the front and rear PM are hinged laterally outwardly, thusly foreshortening their effective longitudinal length, i.e. their length as projected onto the longitudinal center line 15. When combining this with the donning and the transformation from an essentially flat configuration into the generally U-shaped configuration (see FIG. 2B, C), this foreshortening lifts the pull means into the anal cleft of a user.

Concurrently with this lifting through the pull means, also the spreading means, and in particular the connecting regions 1303 and 1307 are lifted up along a line extending through the groin clefts rearwardly across the buttocks. This induces a cross-directional pull force along the SM strips 1312 and 1318, which further transmit this pull force to the discontinuity of the openings, which consequently open cross-directionally, thereby creating an even further foreshortening effect, albeit to a lesser degree than that of the front and rear PM.

Thus, comparing FIGS. 3A and 3B, the longitudinal distance rear margin points A to front margin points F is significantly reduced in the in-use configuration, whilst the distance between points B and E is only slightly shorter.

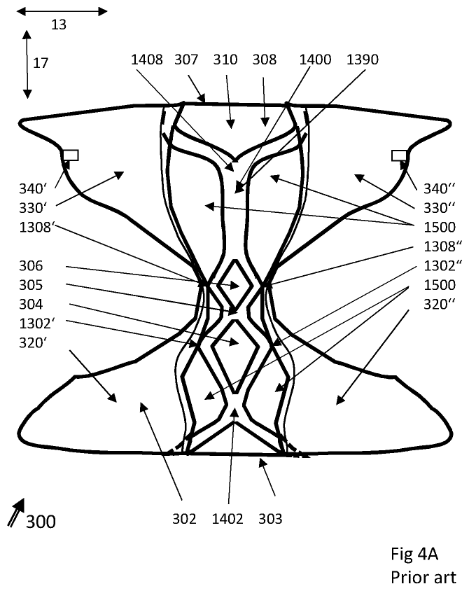

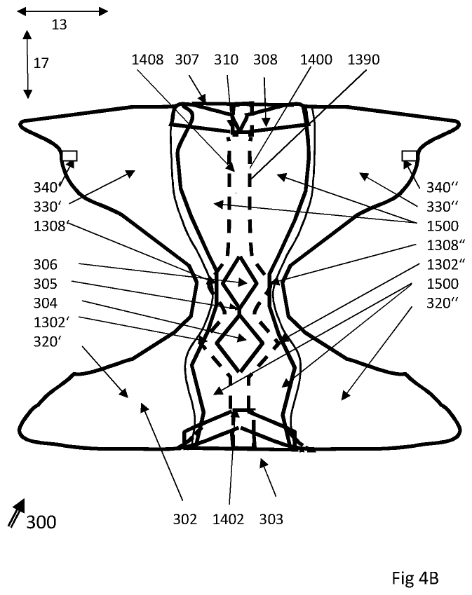

Referring to a particular execution as shown in FIG. 4A, the overfolded portion may also be directly connected to the periphery of the opening, thereby directly inducing the spreading effect upon donning. FIG. 4B depicts a similar article, wherein the PM is positioned underneath. i.e. away from the wearer, relative to the SPS.

It should be noted that this is a purely geometric effect, and does not require extensibility or elasticity of the employed materials. Generally, within the present context, a material is considered non-clastic, if upon application of a strain corresponding to regular manufacturing conditions, it does not extend in its machine direction by more than 5%, preferably not more than 2% relative to its metered-in length.

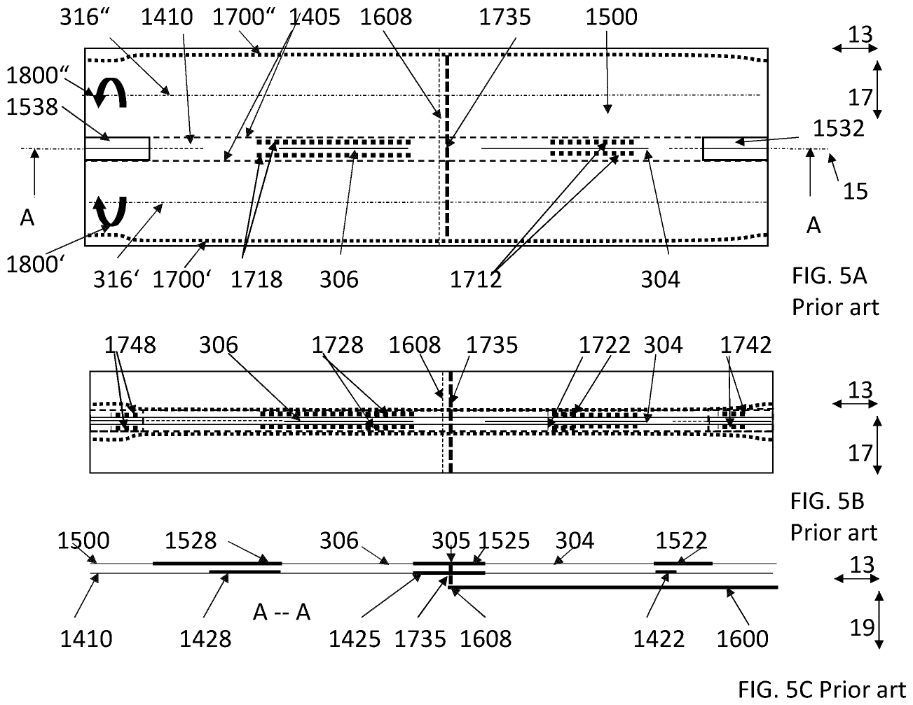

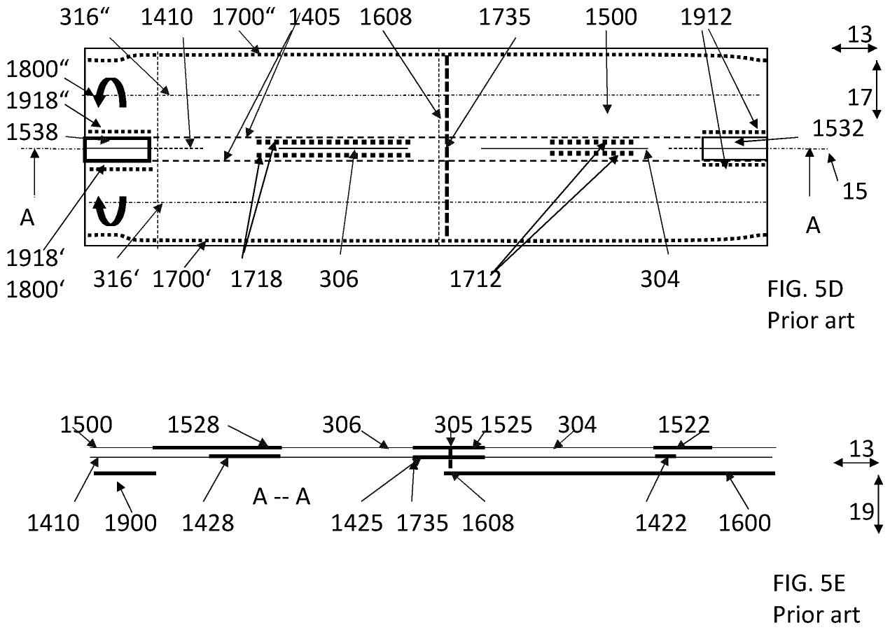

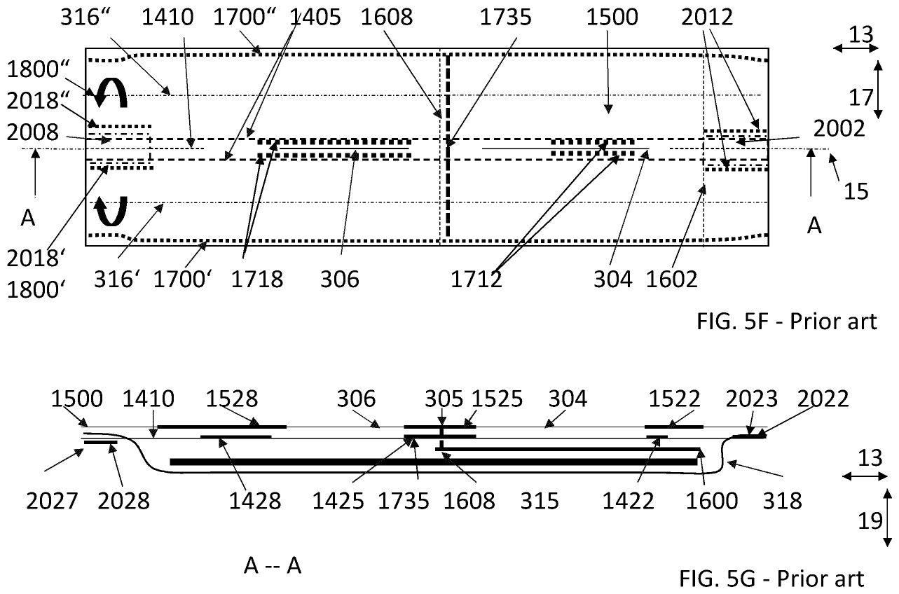

It is not essential, whether the PM is positioned z-directionally on top of (i.e. towards the wearer) or underneath (i.e. away from the wearer) the SPS. FIG. 5A to C depict schematically and exemplarily a design with FIG. 5A showing a view onto the SPS 1500 prior to overfolding but already connected to the center piece (not visible) by connecting lines 1700. FIG. 5B after overfolding (indicated in FIG. 5A by folding arrows 1800) along folding lines 316, and FIG. 5C a cross-sectional view AA of the unfolded article along the center line 15. The SPS 1500 extends over the full length and is separated. e.g. cut, along the center line 15 over the full length except for three non-separated sections 1522, 1525, and 1528, respectively. Further, a front (1532) and a rear (1538) cut out in the SPS, preferably as shown at a width of about the width of the PM strip 1410, is provided towards and reaching into the front (303) and rear margins (308), to allow proper bonding of the side margins to the thusly exposed PM after the overfolding. Further shown in FIG. 5A is a pull means strip 1410 underneath the SPS with its longitudinally extending side margins 1405 shown as dashed lines. Also the pull means strip 1410 is separated along the longitudinal center line 15 except for three non-separated sections 1422, 1425, and 1428 (see FIG. 5C). Further. ESS 1600 is positioned in the front portion underneath the SPS and PM, with at least its front margins coinciding with the ones of the SPS and its rear margin 1608 shown as dotted line in FIGS. 5A and B just rearwardly of the crotch point 305.

The PM and the SPS are connected to each other in the front and rear (1712 and 1718, respectively) laterally outwardly of certain sections of the front (304) and rear (306) opening. The first surface of the SPS is connected to the overfolded portion to enable the spreading at front (1722) and rear (1728) SPS overfold connections. Further, the overfolded portion is connected to the PM strip through the front (1532) and rear (1538) cut out in the SPS at overfold-to-PM connections 1742 and 1748, respectively. Upon donning, the article will unfold as described in the above and shown in FIG. 2A, except that now the SPS is covering a major part of the PM strip, thereby giving a more pleasing appearance.

In order to avoid potential leakage in the areas of front and rear cut outs 5132, 1538, the ESS may extend from its crotch point margin 1608 forwardly towards and beyond the front margin of the SPS, and may during manufacturing extend into the rear margin of the preceding article. As illustrated in FIGS. 5D and E, this can be achieved by adding front masking connections 1912 to connect the ESS to the SPS along front masking connections 1912. Similarly, in the rear portion, a masking sheet 1900—as may be added separately but preferably is generated during manufacturing by extending the ESS as described above beyond the front margin of the SPS—may be added and connected to the SPS aside rear cut out 1538 by cut out connections 1918.

Whilst such a design functions quite well, an even further improvement can be achieved by adapting the article to be combined with a suspender system separate from the article.

To this end, and when starting from an article as described in connection with FIGS. 5F and G, the fixation means 2028 are not connected laterally outwardly to the article, but they are replaced by suspender connection means, which are adapted to be used with a suspender system.

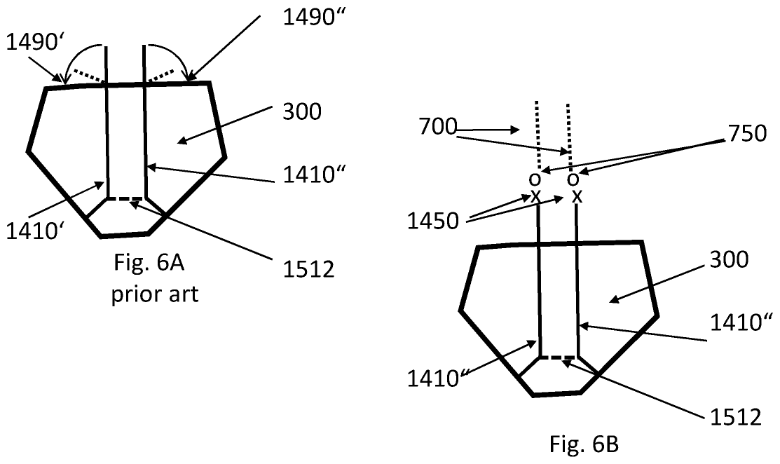

Thus, similar to the execution of FIGS. 5F and G, the pull means comprises at least one pull means strip, that extends from the front portion through the crotch portion to the rear portion of the article and further comprises suspender connection means. These are elements that allow connecting the pull means strip with a suspender system that is separate from the article. This is to be seen in contrast to the executions of PCT 716, as depicted schematically in FIG. 6A. As generally described in the above, the article 300 comprises an SPS 1512, which is shown as being lifted upwardly (i.e. towards the body of a wearer) by pull means 1400 with a first and a second (e.g. left and right) pull means strip 1410′ and 1410″, respectively, that are pulled out and then connected at the laterally outwardly positioned connection points 1490′ and 1490″, respectively.

In contrast thereto, the pull means strips 1410 according to the present invention, as depicted in FIG. 6B, comprise suspender connection points 1450, that are adapted to be connected to a suspender system 700, as will be described in more detail herein below, with connection points 750.

A suspender system in the present context refers to elements that are adapted to be worn on the body of a wearer and support the maintenance of the positioning of the pull means and SPS. In a first execution, a suspender system may be a girdle around the waist of a wearer. In another execution, a suspender system may be adapted to be worn over the shoulders of a wearer, also referred to as braces. In yet a further execution, the suspender system may be adapted to be worn around the neck of a wearer and extend to the front and/or rear waist of the wearer.

It is important, that the suspender system adapted to be connected to the pull means strip functions independently from fit sustaining elements of the article. i.e. the article has to be adequately maintained on the body even if no suspender system is present, as satisfied by most conventional articles to a certain degree, and in particular by articles according to the teachings of the above referenced EP2503973B1. Thus, there are essentially two independent force line systems—one for maintaining the article on the body of a wearer, typically running around the hips or the waist of a wearer, such as known from conventional articles, but especially from articles as described in PCT 716, and another one separate from the article for selectively pulling the pull means and SPS. It is not critical how the force lines of the suspender systems run—but together with the pull means strip system they need to encircle portions of the body in a closed force line (loop) around waist. shoulders or neck of a wearer, i.e. they partially run around portions of the wearer's body that are outside the article.

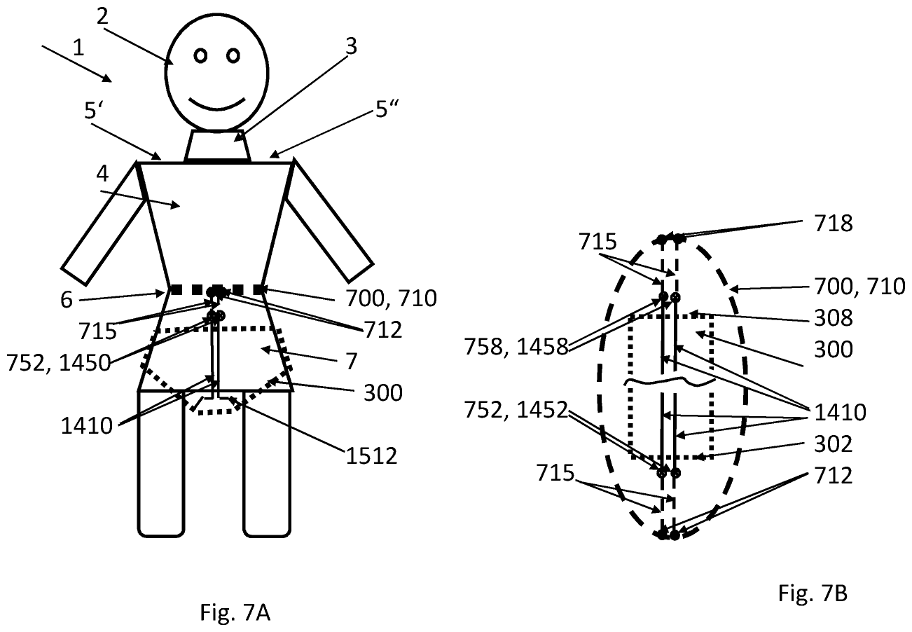

For further explaining the principle, typical, though not limiting, executions for such suspender systems are depicted in FIGS. 7 to 9 with a wearer 1, his head 2, neck 3, chest 4, shoulders 5 with a first and a second, e.g. left and right shoulder 5′, and 5″, waist 6, and lower torso 7. As shown, the wearer faces the viewer with his front side.

Thus, FIGS. 7A and B depict a waist girdle 710 positioned in the waist portion 6 of a wearer as a particular execution of a suspender system 700, with waist girdle suspender straps 715 connected via front girdle to girdle straps connection points 752 of the suspender straps to the suspender connection points 1450 of the pull means strips 1410 of the article 300, which is positioned below (relative to the wearer) the waist girdle 710. FIG. 7B depicts schematically the front 302 and rear 308 portions of an article 300 (dotted line) with pull means strips 1410 (indicated as solid lines) connected via front (1452) and rear (1458) releasable connections to the suspender system (indicated as “x”) to suspender straps 715 (dashed line) with releasable connections 752 in the front region and 758 in the rear region (indicated as “0”) to the pull means strips 1410 and being connected, preferably permanently, (indicated as solid dots) to the waist encircling girdle 710 (dashed line) at front and rear girdle connection points 712, 718, respectively. The size of the waist girdle can be adjusted by conventional means (not shown). In an alternative embodiment, the pull means strip section may not be separated in two strips 1410, but be connected as a single piece.

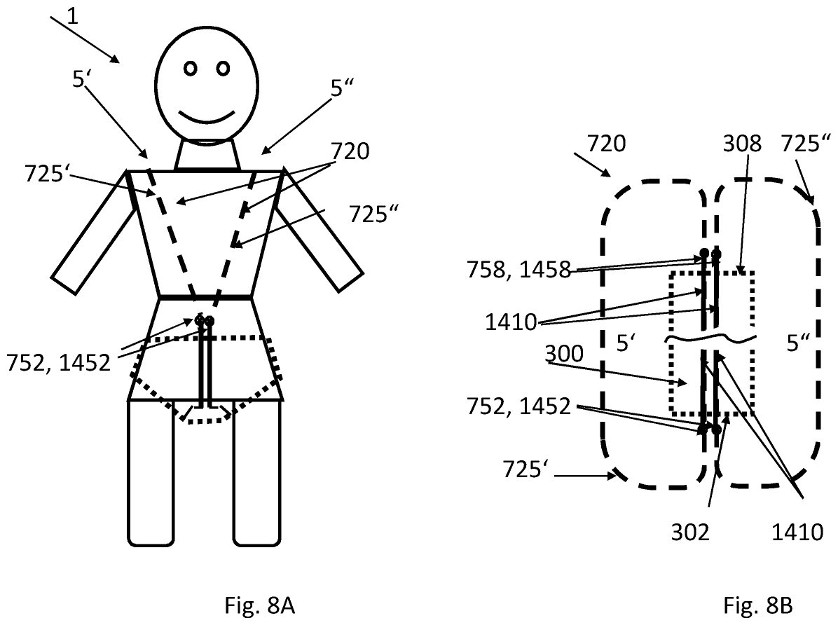

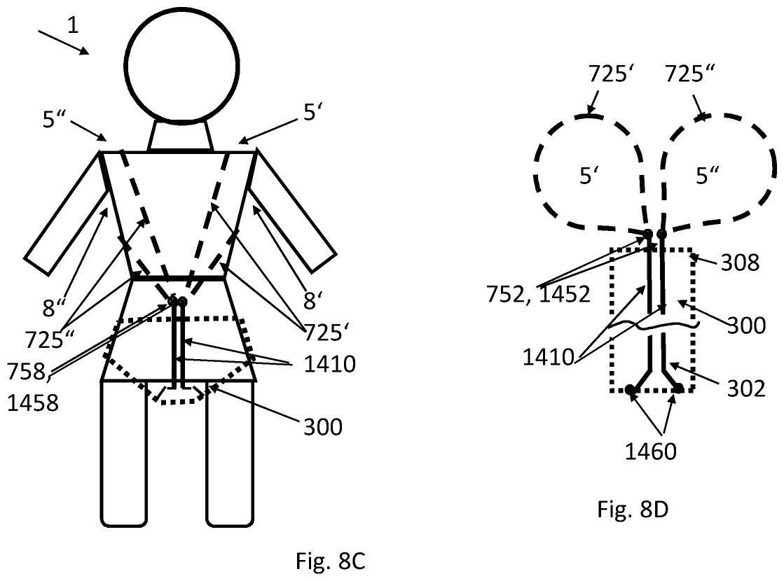

Similarly, in FIGS. 8A and B a shoulder strap or braces system 720 is depicted wherein the strips 725′ and 725″ of the braces run over the shoulders 5′ and 5″ of a wearer 1 and are connected with shoulder strap connections 752 and 758 both in the front (302) and rear (308) portion of the article 300 to the suspender connection points 1452 and 1458 of the pull means strips 1410. The length of the braces strips can be adjusted by conventional means (not shown).

In a variant of this execution shown in FIGS. 8C and D with a view to the back of a wearer, the strips 725 are running over the shoulders 5′ and 5″ and underneath the armpits 8′ and 8″ such that they are connected to the pull means strips 1410 in the rear portion 308 of the article only. In the front portion of the article, the pull means strips are connected to the article at connection points 1460, as described in PCT 716.

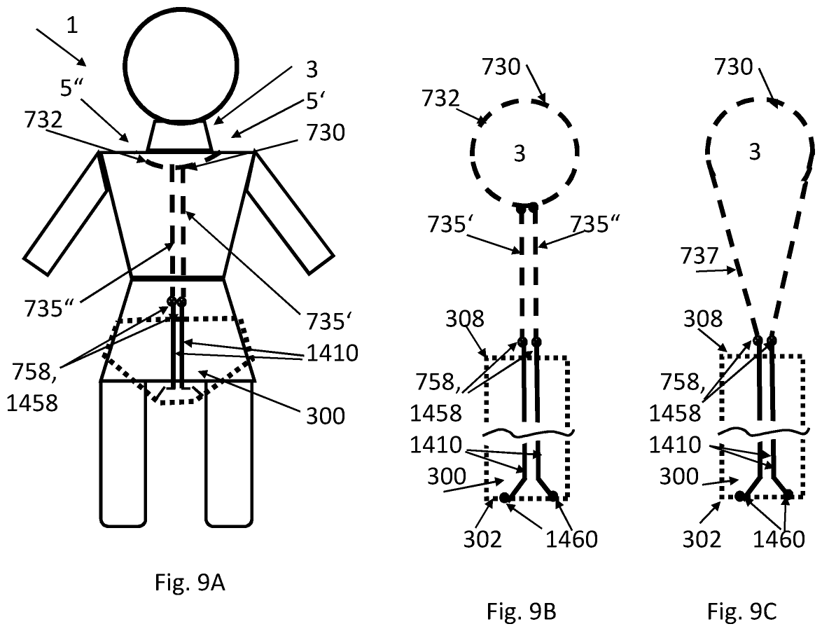

A further execution is depicted in FIG. 9A to C, also showing the back view of a wearer, with neck holder strap system 730. Similar to the execution shown in FIGS. 8C and D, the suspender straps are refastenably connected to the pull means strips in the rear portion 308 of the article 300 only. As depicted in FIGS. 9A and B, two strips 735 are connecting a neck surrounding strip 732 with the pull means strip 1410. Alternatively, as depicted in FIG. 9C, a single strip 737 may be running around the neck.

The suspender system may be of any material that is sufficiently strong and soft to withstand the pull as may be transferred when the pull means is activated, i.e. pulled upon donning. Thus suitable materials include nonwoven materials as also suitable for being used in the article.

It is also not critical for the functioning of the present invention, if the suspender system is intended for single or multiple use, the latter being preferred for cost and environmental (disposal) reasons, or for a limited re-usability. i.e. the suspender system can be re-used until it is soiled. e.g. after several uses, referring to more than 2 but less than about 20 changes of the corresponding article.

In a preferred execution, the suspender system is made of breathable material so as to avoid occlusion of the covered skin. Preferably, the suspender system is not elastic, but allows to be adapted to the size of the wearer. e.g. via adjustable length or circumference. More preferably, the adapting can be performed upon donning of the suspender system simultaneously with the donning of the hygiene article.

Further, the suspender systems and the disposable article should be adapted to each other so as to provide a matching kit, comprising connection systems that allow connecting of the suspender system with the respective counter-elements of the pull means strips of the article, namely the suspender connecting means of the pull means strips.

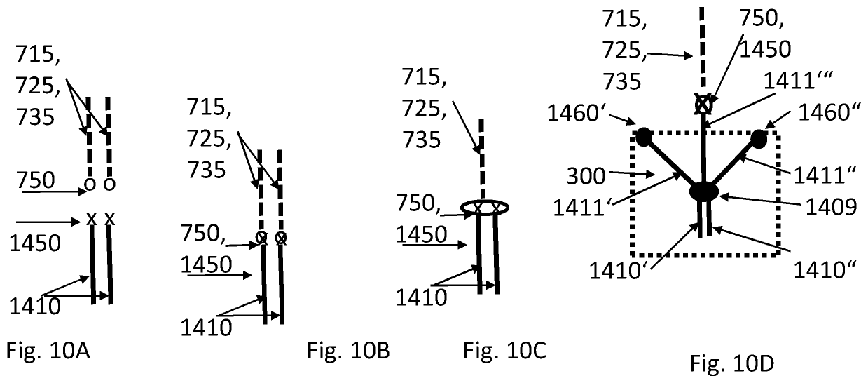

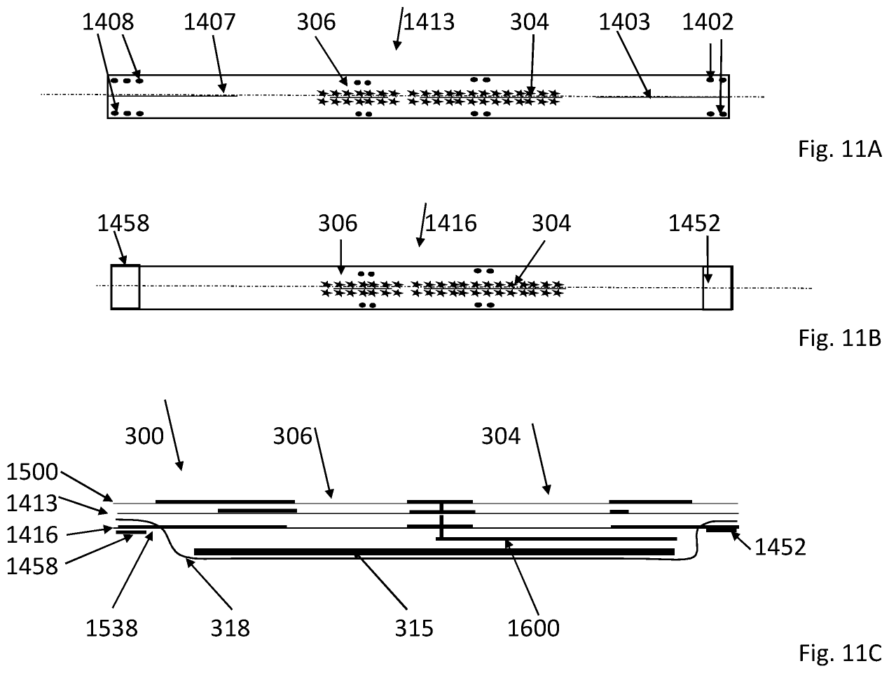

FIGS. 10A and B depict schematically how connection elements 750 of the pull means strips (depicted as “x” at the ends of the full lines) match connection elements of the suspender systems 1450 (depicted as “o” at the ends of dashed lines), which may be positioned in the front (denoted as 752 and 1452, respectively) or rear portions (denote as 758 or 1458, respectively) of the article or in both, with FIG. 10A describing a “pre-donning” configuration, and FIG. 10B an in-use configuration. Whilst in the previous description the pull means strips and the suspender straps have been shown as two separate, often parallel strips, the skilled person will readily realize that for certain executions the strips may be single strips, or two parallel stipes may connect with a single strip, as indicated in FIG. 10C. A further execution as shown in FIG. 10D comprises two pull means strips, that are connected to themselves at a connection point 1409 to the split into a first and a second strip section 1411′, and 1411″, respectively, that are connected to the article near the rear or front margin outwardly of the longitudinal center line. A further strip section 1411′″ is also connected to the other two at the connection point 1409, further comprising the connection means at its other end so as to allow connecting to the suspender system, here shown by a single suspender strap 705. FIG. 11 refers to a combination of a system with a first pull means permanently connected to the article with a pull means not connected to the article but adapted to be connected to suspenders. In FIG. 11A, a first PM strip 1413 is shown, corresponding to PM strip 1410 as in FIG. 3A, with a front (304) and a rear (306) opening with respective connections, front (1403) and rear (1407) discontinuity, and front and rear connection points 1408 and 1402, respectively, for permanently connecting the PM strip to the lateral portions of the article. Upon donning, these spread the discontinuities, as described in the context of FIG. 4. The spreading of the discontinuities depends on the lateral distance of the connection points to the longitudinal centerline, and for certain designs it might be desirable to have a wider spreading, in order to have more pull of PM and SPS into the anal cleft. To this end, a secondary PM strip 1416 is positioned parallel to the first PM strip and preferably also over the full length of the article. Front and rear openings 304 and 306 are in registry with the ones of the first PM strip, and so are the connecting points in the crotch region. Towards the ends, the secondary PM strip does not necessarily comprise the discontinuity, but has suspender connection points 1450. FIG. 11C depicts a cross-sectional view of an article comprising the first (1413) and secondary (1416) PM strips. Relative to the first PM strip, the secondary one is positioned towards the backsheet side of the article, and runs through front (1532) and rear (1538) cut out of the SPS, such that the suspender connection points of the secondary PM strip can be readily connected to the suspender system (not shown in FIG. 11), thusly allowing to adequately adjust the pull means to the wearer.

Whilst the connecting between the PM strips and the suspender system may be achieved by a multitude of connecting systems, such as buttons, snap-fastener, macro-fastener (as described as “slot and tab fastener” in EP1009350A1), or even knotting, a particularly preferred execution relates to the use of mechanical fastener systems, such as well-known in the art and commercially available e.g. from 3M, (US), Aplix (France), or Bender (Germany). Either of the “male” hook elements or the “female” loop elements may be on the article or the suspender side. As typically the male hook elements are stiffer and harsher than the female loop elements, it is preferred that they do not have direct contact with the skin of a wearer, such as by being oriented outwardly.

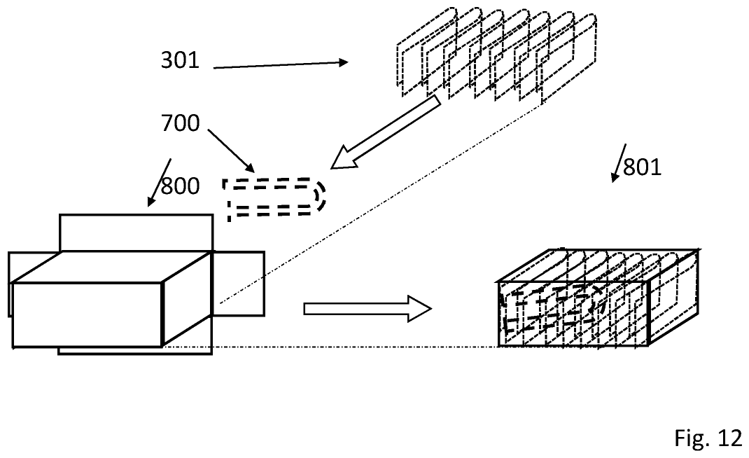

In a further aspect and as schematically depicted in FIG. 12, the present invention is a combination package 800 with an array of more than one of such disposable articles 300 combined with a different, smaller number of semi-durable suspender systems 200 into a filled package 801. Such suspenders systems are not necessarily intended to be re-used, such as by being washed, but can be used for several changes of the disposable articles. Thus, a package of disposable articles may contain one or more suspender systems, but less than disposable articles, such as a fifth of the number of the latter. The skilled person will also appreciate that any further exudate handling means, such as liquid or facces acquisition features, may be incorporated into or added to the absorbent core or be positioned between the absorbent core and the SPS or ESS.

All discontinuities may also be executed as partly separated lines, where the separation is incomplete, such as by an intermittent cutting line or a perforation line (sometimes also referred to as “perf 'n pop”), such that the material remains connected during at least a part of the process, but is readily separated at least upon donning and in the in-use configuration. For the front and rear openings the x-y-extension will be formed from the respective separation lines upon the transition from the manufacturing configuration into the in-use configuration. Whilst the discontinuity may be and often preferably is executed as a separation line, such as a cut, it may also be formed by removing material from the web so as to form an opening already at this point in the manufacturing configuration. The connecting of the various elements may be achieved by any conventional means, such as without limitation heat or pressure bonding, or sonic, preferably ultrasonic bonding, though especially for the connections involving the overfolded regions, use of appropriate glues is preferred from a processability point of view.

As a skilled person will readily realize, the term “point”, e.g. “connecting point”, may exhibit a certain size, e.g. corresponding to the applied technology for connecting. Thus a glue-type connecting point includes a patch of glue sufficiently small to not impact functionality in the surrounding region. Similarly, the term “line”, e.g. “connecting line” may exhibit a certain width. Also the term encompasses an interrupted line, e.g. comprising a dotted glue or ultrasonic bonding line, as well as a bonding pattern, such as a line made of a multiplicity of (sub-) lines or a connecting point made of a multiplicity of smaller bond points. Similarly, the term “region” encompasses a long but narrow region that may also be seen as a “line”.

The width of the CPSM has to be at least the overfolding gap width as the distance of the overfolded side margins to each other, plus the machining tolerance for positioning and connecting the CPSM to the overfolded regions. This machining tolerance may be very small, e.g. less than about 5 mm, but should not exceed about 10 mm or 20 mm for material usage efficiency reasons. Generally, the SPS, PM, CPSM, or SM can be made from a broad range of raw materials satisfying the general requirements that apply to hygiene articles to be worn on the lower torso of a wearer, such as not compromising on comfort or health aspects. Routine adaptation to the specific application will determine strength, softness, air- and liquid permeability, etc., of the materials. Particularly when the articles are intended for large scale production, the materials are preferably web materials. Generally, the term “web” relates to any material which is essentially endless or continuous in one direction (generally denoted as “x-direction” or “machine direction”). Webs are often, but not necessarily, stored, supplied or used in roll form and thusly also sometimes denoted “roll goods”. Whilst these are then not “endless” in the strict sense of the word, their extension in this x-direction is significantly larger than in any other direction. By combining consecutive rolls or other batches, (“splicing”) such webs can be considered “endless” for all practical purposes. Webs may be transported in a “batch” form, such as when a roll thereof is shipped, or they may follow a “web path”, such as when the webs are unwound from a roll, as described hereinafter. Typical examples for webs are—without implying any limitation—plastic films or foils, optionally apertured, textiles, non-wovens, nets, or scrims.

The SPS and the pull strip materials are preferably compliant, soft feeling, and non-irritating to the wearer's skin and may be manufactured from a wide range of materials, such as porous foams; reticulated foams; apertured plastic films: or woven or nonwoven webs of natural fibres (e.g. wood or cotton fibres), synthetic fibres (e.g. polyester or polypropylene fibres), or a combination of natural and synthetic fibres. If they include fibres, the fibres may be spunbonded, carded, wet-laid, meltblown, hydro entangled, or otherwise processed as is known in the art. They may be a composite material, such as when comprising an open net or scrim structure in combination with a spunbonded web.

Preferably, the SPS exhibits a low tendency for the passage of facces. Optionally, the SPS may exhibit a z- or thickness directional gradient structure, or be a laminate or composite material, such as exhibiting particularly skin friendly properties on the user oriented surface, or particular faeces absorbent properties on the opposite surface, which may be particularly beneficial in the context of low viscosity or “runny” facces.

Optionally, the SPS may be a composite material, such as being made in strips (y-directional variation) or connected patches (x-directional variation).

Optionally, the SPS may comprise additives, such as skin friendliness enhancers, such as emollients or the like, as well known in the art.

For executions where the SPS extends more forwardly into regions of urine or menses loading without a front opening, it should exhibit—at least in these regions—good liquid permeability, such as by being hydrophilic by nature of the materials employed, or by treatment. For executions comprising a front opening allowing exudates to pass through, it may be preferred that the SPS is fluid impermeable, e.g. as a hydrophobic nonwoven, a film, or a composite thereof.

A kit comprising a hygiene article and a suspender system, preferably a re-usable suspender system, is particularly suited for being combined with a sensor system comprising sensor elements that can detect physical, chemical or biological characteristics of the article as is or as loaded with exudates.

A physical characteristic of the article as such may be the strain as applied to or by the pull-band, be this upon donning of the article, or during use, such as when the positioning of the article changes.

Such a strain may well be detected by strain gauges, as well known in the art for other applications.

Further, the sensor element may detect temperature changes, as may indicate a urination or defecation, or body temperatures as such, e.g. during illness. A chemical or biological sensor may detect contents of the exudates, e.g. urine, such as vitamin C, leukocyte, urease, bilirubin, occurred blood, nitrite, pH value, protein, urine specific gravity, ketone body, glucose, micro-albumin content.

A physical sensor element may be combined with a chemical or biochemical sensor, e.g. when a urine detection test paper reacts with a colour change and the colour detecting sensor transmits the colour change into an electrical signal, as known in the art, e.g. from CN113932803, to which express reference is made with regard to the conversion of the urine content analysis into an electrical signal.

The sensor element transforms the detected characteristic into an electrical signal. To this end, the sensor system further requires an electrical energy supply, such as a battery, which is connected to the sensor elements via a cable.

The energy supply may be rechargeable, and is preferably connected to or integral with the suspender straps, which may be executed as reusable straps, that preferably allow cleaning, e.g. by washing.

The electrical connecting may be suitably integrated into the suspender connection means, whereby the electrical connections is executed so as to avoid any potential dangers to the user, be this the caretaker or the wearer. Such an electrical connecting between disposable article and the suspender straps is not needed, if the sensor is integrals or connected to the suspender straps, as may be a preferred execution for the strain gauges as sensor element.

The sensor system further comprises a signal transmitter element, connected to or optionally integral with the energy supply. The transmitter element transforms the signal into an electromagnetic signal that can be received wirelessly by a user, such as, but without limitation, a Bluetooth signal as well known in the art, and which may be received on a receiver, such as—also without limitation—a mobile phone, to be translated there into a visual or acoustic information to the user. Optionally, a computer program may perform further analysis of the data, so as to ease interpretation by the reader.

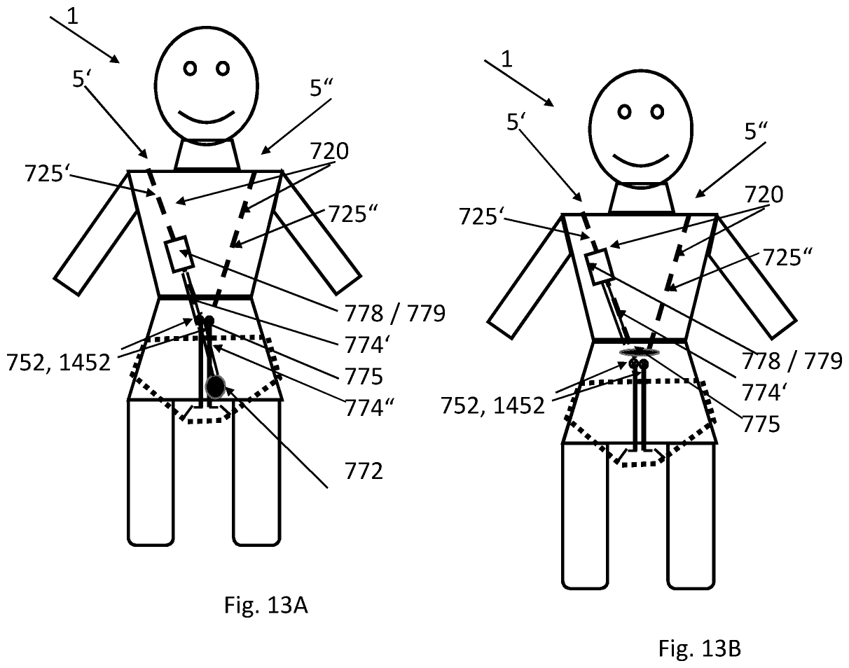

The principle of adding a sensor system to a kit of a hygiene article and a suspender system is now further explained by referring to FIG. 13A, wherein exemplarily a kit comprising a disposable diaper 300 and a suspender system 700 as depicted and described in the context of FIGS. 8A and B is shown, further complemented by a sensor system with a sensor element 772 positioned in the disposable diaper, a cable 774 connecting via connector 775 at the connection points 750 of the suspender straps 735 with an energy supply 778. A transmitter 779, as shown integral with the energy supply is adapted to transmit the signal to a receiver 780.

FIG. 13B depicts a different execution of a sensor system, wherein a strain gauge sensor element 773 is connected or integral with a suspender strap, to which the energy supply and transmitter may be connected, or which may be integral there with.

1. A hygiene article for being worn on the lower torso of a wearer adapted to receive and retain bodily exudates,
said article exhibiting:
a length/longitudinal/x-direction and a longitudinal centerline,
a width/cross-directional/y-direction, perpendicular thereto and corresponding to the left-right direction of the wearer,
a thickness or z-direction perpendicular to both;
said article comprising, relative to a wearer during its intended use,
a rear waist region and a front waist region, and
a crotch point region positioned longitudinally between said waist regions
and comprising a crotch point positioned between the anal opening and the genital organs of a wearer during its intended use,
said article being adapted to be converted
from a manufacturing configuration
into an in-use configuration,
wherein said article adopts a general U-shape by said longitudinally extending center line extending from the front or rear waist region of a wearer through the crotch point region into the opposite waist region of the wearer,
and wherein said front and rear waist regions are adapted to encircle the waist of a wearer,
said article comprising
a skin protection sheet (SPS)
adapted to separate bodily exudates from the skin of a wearer,
and comprising at least one opening adapted to be positioned in registry with a bodily exudate releasing body opening or a genital organ,
extending from said front or said rear waist regions at least into the crotch point region of said article, optionally into the opposite waist region;
a backsheet adapted to retain said bodily exudates,
wherein at least one of said SPS and said ESS, if present, comprise(s) longitudinal side margins that are overfolded along a longitudinal fold line towards but not over said longitudinal center line of said article,
said article further comprising
a spreading means (SM) adapted to maintain at least one of said opening(s) cross-directionally open during the article's intended use,
a pull means (PM) comprising
at least one longitudinally extending pull means strip(s),
one or more suspender connecting means adapted to be connected to a suspender system which is connectable to but separate from said article.

2. A hygiene article according to claim 1, wherein said suspender connecting means of said PM strip(s) is/are positioned at the front and/or rear ends of said pull means strip strip(s).

3. A hygiene article according to claim 1, wherein said suspender connecting means of said PM strip(s) is/are positioned in the front and/or rear waist region.

4. A hygiene article according to claim 1, wherein
said at least one pull means strip extend(s) from at least one of said cross-directionally extending margins in the front or rear waist region of said SPS or ESS towards said crotch point,
if covering said opening(s) of said SPS, further comprises (a) pull means opening(s) in registry with said openings(s) of said SPS.

5. A hygiene article according to claim 1, wherein said spreading means (SM) being unitary with said pull means,
wherein said spreading means and said PM are
partially cut from said pull means by a separation line
extending
from and intersecting a longitudinal side margin of said pull means
towards but terminating at a termination point before intersecting said pull-means opening,
whereby
said intersection of said longitudinally extending side margin
is distanced further away from said crotch point
than the termination point of said separation line;
and connected at its laterally outward end to said overfolded portions of said SPS or ESS; or
a reinforcement means adapted to maintain a pre-cut opening cross-directionally open, optionally comprising an attachment means, optionally of the skin adhesive type.

6. A hygiene article according to claim 1, wherein said PM comprises
a first pull means strip permanently connected to said article in at least one of said waist regions;
a second pull means strip positioned z-directionally adjacent to said first pull means strip comprising suspender connection means.

7. A kit of a hygiene article according to claim 1 and a suspender system.

8. A kit according to claim 7, wherein said suspender system is selected from the group consisting of
a waist girdle;
a braces system running over the shoulders of a wearer;
a neck holder system,
whereby each of these systems are connected to the article by suspender straps that are adapted to be refastenably connected to said suspender connecting means of said pull means strips of said article.

9. A kit according to claim 7, further comprising a sensor system comprising
a sensor element;
an energy supply;
a connecting element;
a signal transmitter,
whereby at least said energy supply and said signal transmitter are connected to or integral with said suspender system.

10. A kit comprising a sensor system according to claim 9, wherein
said sensor element is adapted to detect physical, chemical, or biological characteristics of said disposable article or of exudates deposited therein.

11. A kit comprising a sensor system according to claim 9, wherein said hygiene article is a disposable hygiene article and said suspender system is a re-usable suspender system.

12. A hygiene article according to claim 1, further comprising one or more elements selected from the group consisting of
side panels,
an absorbent core adapted to absorb liquids of said bodily exudates,
an exudate separation sheet (ESS) adapted to separate exudates from each other or from predetermined portions of said article;
said pull means (PM) comprising at least one longitudinally extending pull means strip(s) comprising PM sub-strips, adapted to urge said SPS into the anal cleft of a wearer during the article's intended use.

13. A hygiene article according to claim 5, wherein said reinforcement means adapted to maintain a pre-cut opening cross-directionally open further comprises an attachment means, optionally of the skin adhesive type.

14. A hygiene article according to claim 13, wherein said reinforcement means adapted to maintain a pre-cut opening cross-directionally open further comprises an attachment means of the skin adhesive type.

15. A kit of a hygiene system according to claim 7, wherein said suspender system is a reusable suspender system.
